(19) United States
(12) Patent Application Publication
(10) Pub. No.: US 2011/0034788 A1
(43) Pub. Date: Feb. 10, 2011

(54) METHODS AND APPARATUS FOR USING MULTIPLE SENSORS TO MEASURE DIFFERENTIAL BLOOD TRANSPORT TIME IN A PATIENT

(75) Inventor: Robert Stoughton, Boulder, CO (US)

Correspondence Address: Nellcor Puritan Bennett LLC;ATTN: IP Legal, 6135 Gunbarrel Avenue, Boulder, CO, 80301, US

(73) Assignee: Nellcor Puritan Bennett LLC, Boulder, CO (US)

(21) Appl. No.: 12/535,311

(22) Filed: Aug. 4, 2009

(51) Int. Cl. A61B 5/1455; A61B 5/02

(52) U.S. Cl. 600/323; 600/504

(57) ABSTRACT

The difference in time that it takes for blood to flow to two locations in a patient's body can be measured by placing, on the patient's body, sensors for non-invasively detecting a variable characteristic of the content of the blood at each of those locations. The time of occurrence of a change in that characteristic as detected by one of the sensors is compared to the time of occurrence of that same change as detected by the other sensor. The difference between these two times is a measure of the difference in time that it takes for blood to flow to the locations of the two sensors.

### SUMMARY OF THE DISCLOSURE

This disclosure relates to monitoring or measuring the time required for blood to flow through various parts of a patient's circulatory system.

There are many diagnostic contexts in which it would be desirable to be able to determine how long it is taking for blood to flow to various parts of a patient's body. For example, the length of time required for blood to flow from the heart to an extremity such as a finger or a toe can be used as an aid in determining the condition of the patient's arteries leading to that extremity. It would, of course, be best if such blood transport time data could be gathered minimally or non-invasively.

Various non-invasive instruments are known for measuring one or more of the variable properties of the content or composition of blood that is flowing through tissue or tissue structures below the patient's skin without physically penetrating the skin. An example of such an instrument is an oximeter. The sensor of an oximeter, such as a pulse oximeter or a regional oximeter, can be attached to an external location on the patient, and it will provide an electrical output signal indicative of the degree of oxygen saturation of the blood passing through the tissue adjacent to where the oximeter has been attached.

As an aid to clarity, the following discussion will refer, for the most part, to examples in which oximeters and their sensors are used for the purposes of this disclosure. But it will be understood that this is only an example, and that any other type of sensor that can remotely (and therefore non-invasively) monitor a variable characteristic of blood flowing in a patient can be used instead of oximetry. The term “non-invasive blood characteristic sensor” or the like will sometimes be used as a generic term for all such devices (and it will be understood, of course, that an oximeter and its oximetry sensor is one example of such a device or sensor).

As a further preliminary matter, it should be understood that the blood “properties” or “characteristics” referred to herein are properties or characteristics of the content (e.g., the chemical and/or physical make-up or composition) of the blood, not such attributes as its pressure. Thus blood pressure is not typically among the blood properties or characteristics employed in accordance with this disclosure. Again, the blood properties or characteristics that are employed herein are blood content or composition properties.

In accordance with certain aspects of this disclosure, at least two non-invasive blood characteristic sensors (e.g., oximetry sensors) are attached to the patient at different locations (e.g., to the forehead and to a toe). Each of these sensors is operated to produce an output (e.g., an electrical signal) indicative of the present value of a variable characteristic of the patient's blood at the location of that sensor. For example, this characteristic may be the degree of oxygen saturation of the blood at the location of each sensor. As an even more specific example, this may be the characteristic conventionally known as SpO₂ in the case of pulse oximeters or SrO₂ in the case of regional oximeters. (The term SxO₂ will sometimes be used herein as a generic term for both SpO₂ and SrO₂). The output signals of both sensors are monitored for the occurrence of a recognizable change in the characteristic being monitored (e.g., a recognizable drop in SxO₂, or a recognizable increase in SxO₂). The time at which such a recognizable change is detected at each sensor is noted. Then the elapsed time between the two times mentioned in the preceding sentence can be used as a measure or indication of blood transport time in the patient (especially, as a measure of differential blood transport time between the locations of the two sensors).

Further features of the disclosure, its nature and various advantages, will be more apparent from the accompanying drawings and the following detailed description.

### BRIEF DESCRIPTION OF THE DRAWINGS

FIG. 1 is a simplified schematic block diagram of an illustrative embodiment of apparatus constructed in accordance with certain possible aspects of the disclosure.

FIG. 2 is a pair of simplified signal waveforms or traces that are useful in explaining certain possible aspects of the disclosure.

### DETAILED DESCRIPTION

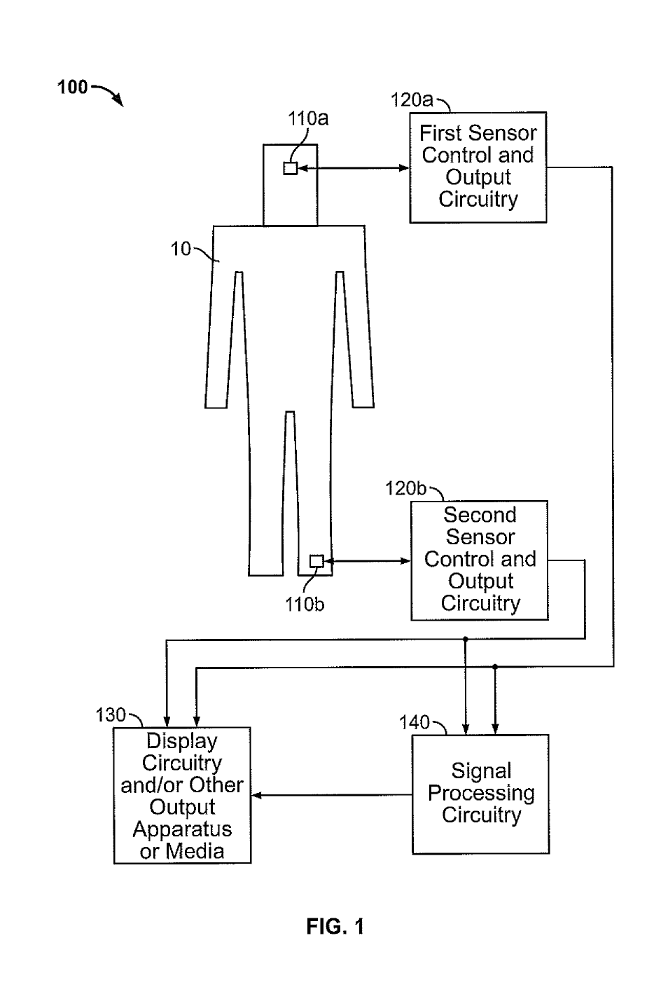

An illustrative embodiment of apparatus 100 in accordance with certain possible aspects of the disclosure is shown in FIG. 1. In addition to showing apparatus 100, FIG. 1 shows a human subject or “patient” 10 who is being studied, monitored, or diagnosed in accordance with the disclosure. It will be understood, however, that patient 10 is not part of apparatus 100, nor is patient 10 part of this disclosure or anything claimed herein. Patient 10 is only shown in FIG. 1 to make what is being discussed herein clearer and more concrete.

In the illustrative embodiment shown in FIG. 1 it may be desired to produce an indication of the length of time it takes for blood in patient 10 to flow to the patient's left foot (e.g., a toe on that foot). Accordingly, a first non-invasive blood characteristic sensor 110a (e.g., the sensor of a first oximeter) is attached to the patient's forehead, and a second, similar, non-invasive blood characteristic sensor 110b (e.g., the sensor of another oximeter) is attached to the patient's left foot toe.

Each of sensors 110a and 110b has associated sensor control and output circuitry 120a and 120b, respectively. For example, each of electrical circuits 120 may apply to the associated sensor 110 the electrical signal or signals needed to operate that sensor. Each of circuits 120 may also obtain from the associated sensor 110 the electrical signal or signals output by that sensor to indicate the current value or level of the monitored characteristic of the patient's blood at the location of that sensor. Each of circuits 120 may at least preliminarily process the associated sensor 110 output signal(s) it receives in order to convert the information contained in such signals to one or more circuit 120 output electrical signals that are more readily used by other circuitry downstream from circuits 120. For example, the output signals of a sensor 110 may be indicative of the intensity of pulses of light at different wavelengths passing through adjacent patient tissue. The associated circuit 120 may analyze these raw light intensity signals to produce from them a more readily usable SxO₂ signal, which then becomes at least one of the output signals of that circuit 120. (It will be understood that terms like “circuit and “circuitry” as used throughout this disclosure may refer to both electronic components (hardware) and the programming of certain electronic components therein (firmware or software).)

The output signals of circuits 120a and 120b may be applied to output display circuitry and/or other output apparatus or media 130. For example, element 130 may be an electronic display for visibly displaying the waveforms of signals output by other components such as circuits 120a and 120b. A clinician user of apparatus 100 can visually observe such a display, interpret that display, and thereby obtain from it desired diagnostic information about patient 10. Component 130 may alternatively or additionally record information of the type described above for display. For example, such recording may be by printing on paper and/or by storage in electronic or in other generally similar form.

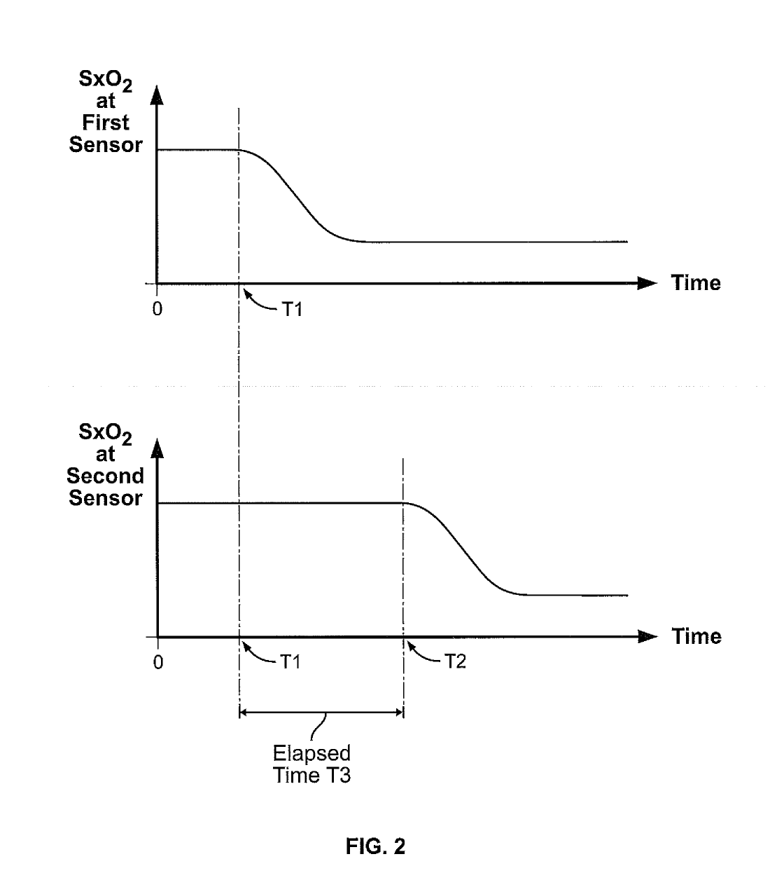

FIG. 2 shows an illustrative embodiment of the type of information that display circuitry 130 may display for observation by a clinician user. In the FIG. 2 example, each of component groups 110a/120a and 110b/120b outputs an electrical signal indicative of the patient's blood SxO₂ at the location of the sensor in that component group. Display circuitry 130 displays a plot or trace of the waveform of each of these signals. As shown in FIG. 2, for example, SxO₂ from the sensor 110a on the patient's forehead is presented in the upper trace on display 130, while SxO₂ from the sensor 110b on the patient's toe is presented in the lower trace on display 130. Both traces are plotted against the same horizontal time scale, with the two traces being synchronized in time with one another (so that any particular instant of time corresponds to a straight line drawn vertically across both traces). Time increases from left to right in these traces. The level, value, or amplitude of each SxO₂ signal is plotted parallel to the vertical axis in each FIG. 2 signal trace, so that a greater value of SxO₂ corresponds to a higher level in the trace.

FIG. 2 shows (upper signal trace) that prior to a certain time T1, SxO₂ measured at the patient's forehead is relatively high. However, at about T1, SxO₂ at that location begins to drop in a manner that can be readily seen and recognized. FIG. 2 further shows (lower signal trace) that prior to another time T2 (which is later than T1), SxO₂ measured at the patient's left foot toe is relatively high. However, at about T2, SxO₂ at the patient's left foot toe begins to drop in a manner that resembles the earlier (T1) drop in SxO₂ at the patient's forehead. The difference in time between T1 and T2 (i.e., T3=T2−T1, or the “elapsed time” between T2 and T1) is a measure of a particular differential blood transport time in patient 10 (i.e. the difference between (1) the time required for blood to flow from the patient's heart to the patient's forehead, and (2) the time required for blood to flow from the patient's heart to the patient's left foot toe). As noted earlier in this disclosure, this kind of information can be helpful in diagnosing various aspects of the patient's condition (e.g., the patient's medical condition).

Returning to FIG. 1, in addition to displaying the above-described SxO₂ signals on display 130, apparatus 100 may include signal processing circuitry 140 for performing (e.g., automatically) various kinds of analysis on the SxO₂ signals in order to aid in the interpretation and use of those signals. For example, circuitry 140 may be able to look for and flag certain kinds of changes in the SxO₂ signals and thereby augment the display of those signals via component 130. As just one illustration of this, circuitry 140 may be able to detect when a significant change in each SxO₂ signal has occurred, and may then cause display 130 to augment its display of the traces of those signals (e.g., by adding chain-dotted vertical lines like those at T1 and T2 in FIG. 2) to the visible display. The presence of such augmentation (e.g., vertical lines) can help the clinician user determine the elapsed time between those display-augmenting features. As still another example of possible capabilities of circuitry 140, that circuitry may actually compute a value for elapsed time T3, and may then cause display 130 to display that value, thereby saving the clinician user from having to determine T3 in some other (e.g., “manual”) way.

The recognizable change in the patient's blood characteristic (e.g., the drop in SxO₂ that begins to show up at T1 at the forehead and at T2 in the toe in FIG. 2) can occur naturally, or it can be induced. For example, sleep apnea causes a patient's SxO₂ to drop significantly. Another way that SxO₂ can be changed is by causing the patient to breathe air having a smaller or larger than normal percentage of oxygen.

Returning to how information obtained in accordance with this invention can be used in patient diagnosis, locating one of sensors 110a on the patient's forehead can be particularly helpful because blood reaches the forehead from the heart promptly and efficiently under most conditions that a patient is subjected to. For example, this typically takes no more than about 10 seconds, and it is not greatly affected by the ambient temperature the patient is experiencing, by whether or not the patient has recently eaten, by whether or not the patient is exercising, etc. A sensor on the forehead therefore provides a good (albeit still somewhat approximate) reference point or time for measurement of blood transport time from the heart to other locations on the patient's body. For example, it can take several minutes for blood that has just left the heart to reach one of the patient's more remote extremities such as a toe. If the forehead is used for T1, and if it is not sufficient to determine T3=T2−T1 as an approximate measure of blood transport time from the heart to the location of the T2 sensor, then a somewhat more accurate measure of heart-to-T2 blood transport time (“HtoT2”) can be determined from HtoT2=T3−TC, where TC is the typical time for blood to flow from the heart to the forehead (approximately 5-10 seconds in almost all cases). It will be appreciated, however, that this disclosure relates primarily to determining differential blood transport time between two locations on a patient's body that typically do not include the heart. Therefore the above references to blood transport time from the heart to an extremity relate only to inferential approximations of such heart-to-extremity blood transport time. The other “differential” blood transport times described herein are direct measurements that do not involve possible inaccuracies due to inferences or approximations.

Although placement of one of two sensors on the patient's forehead is a good approach for many kinds of diagnostic procedures, other procedures may call for different placement of the sensors. For example, if it is desired to compare blood transport time to a patient's two hands, then one sensor 110 may be placed on each hand. Similarly, if it is desired to compare blood transport time to a patient's two feet, then one sensor 110 can be placed on each foot.

This disclosure is not limited to use of only two sensors 110 and associated circuitry 120, etc. For example, three sensors may be used (e.g., one on the forehead and one on each foot to enable simultaneous determination of differential blood transport time to the forehead and to each foot).

Data gathered from a patient in accordance with this disclosure can be used by itself in diagnosis of the patient, or it can be compared to data gathered similarly from other human subjects as a further aid to diagnosis of the patient. For example, the methods and apparatus of this disclosure can be applied to one or more “healthy” patients to collect data as to what various differential blood transport times “should be” (i.e., “expected,” “normative,” “normal,” “reference,” etc., differential blood transport times). Then a differential blood transport time measured for a patient in accordance with this disclosure can be compared to such reference data to determine how much this patient deviates from normal. Such comparisons to reference can be done manually. As an alternative, display 130 may be programmed or otherwise adapted to display such reference data. As still another alternative, signal processing circuitry 130 may be adapted to automatically determine the patient's difference from reference, and to produce an electrical output signal that causes display 130 to display that difference from reference. As another example, a specific patient may have his or her differential blood transport time measured under various conditions (e.g., at rest, immediately after exercise, after a change in body or extremity temperature, etc.). Such measurements may then be used as an individual baseline, against which future measurements are referenced. This may prove useful to measure the efficacy of disease treatment or physical training.

It will be understood that the foregoing is only illustrative of the principles of this disclosure, and that various modifications can be made by those skilled in the art without departing from the scope and spirit of the disclosure. For example, although oximeters have been mentioned for the most part as providing the sensors in the above discussion, it will be understood that oximeters are only an example, and that any other suitable type or types of non-invasive, variable blood content characteristic sensors can be used instead of oximetry sensors if desired. As another example of possible modifications, SpO₂ and SrO₂ (generically SxO₂) are most frequently mentioned above as a variable blood content characteristic that can be non-invasively detected by sensors in accordance with this disclosure; but again, SxO₂ is only an example, and any other non-invasively detectable, variable, blood content characteristic can be used instead if desired.

What is claimed is:

1. A method of measuring differential blood transport time in a patient comprising:
placing a first sensor adjacent a first location on the patient's body;
placing a second sensor adjacent a second location on the patient's body;
operating the first sensor to detect a variable characteristic of the patient's blood at the first location;
operating the second sensor to detect the variable characteristic of the patient's blood at the second location; and
determining elapsed time between when a change in the variable characteristic is detected by the first and second sensors at the first and second locations, respectively.

2. The method defined in claim 1 wherein each of the sensors comprises an oximetry sensor.

3. The method defined in claim 1 wherein the variable characteristic comprises SxO₂.

4. The method defined in claim 1 wherein the first sensor is placed adjacent the patient's forehead.

5. The method defined in claim 4 wherein the second sensor is placed adjacent an extremity of the patient other than the forehead.

6. The method defined in claim 1 wherein each of the first and second sensors comprises a non-invasive blood characteristic sensor.

7. The method defined in claim 1 wherein the characteristic is a characteristic of the content of the patient's blood.

8. The method defined in claim 1 wherein each of the first and second sensors produces a respective one of first and second electrical output signals indicative of variation of said characteristic as detected by the respective one of said sensors.

9. The method defined in claim 8 wherein the determining comprises:
electrically processing said first and second output signals to facilitate determination of said elapsed time.

10. Apparatus for measuring differential blood transport time in a patient comprising:
a first sensor adapted for placement adjacent a first location on a patient's body and operable to produce a first electrical output signal indicative of a variable characteristic of the patient's blood at the first location;
a second sensor adapted for placement adjacent a second location on the patient's body and operable to produce a second electrical output signal indicative of the variable characteristic of the patient's blood at the second location; and
circuitry for outputting indications of the first and second signals in a way that enables determination of elapsed time between when a change in the variable characteristic is detected by each of the first and second sensors.

11. The apparatus defined in claim 10 wherein each of the sensors comprises an oximetry sensor.

12. The apparatus defined in claim 10 wherein the variable characteristic comprises SxO₂.

13. The apparatus defined in claim 10 wherein the first sensor is adapted for placement adjacent to the patient's forehead.

14. The apparatus defined in claim 13 wherein the second sensor is adapted for placement adjacent an extremity of the patient other than the forehead.

15. The apparatus defined in claim 10 wherein each of the first and second sensors comprises a non-invasive blood characteristic sensor.

16. The apparatus defined in claim 10 wherein the characteristic is a characteristic of the content of the patient's blood.

17. The apparatus defined in claim 10 wherein the circuitry for outputting comprises:
a display for displaying waveforms of the first and second signals.

18. The apparatus defined in claim 17 wherein the display displays the waveforms so that time differences between occurrences in the first and second signals can be observed via the display.

19. The apparatus defined in claim 10 wherein the circuitry for outputting comprises:
signal processing circuitry for detecting a similar change in each of the first and second signals, and for determining a difference between time of occurrence of said change in the first signal and time of occurrence of said change in the second signal.

20. The apparatus defined in claim 19 wherein the signal processing circuitry is adapted to produce a time difference electrical output signal indicative of said difference.
